(19) United States
(12) Patent Application Publication
(10) Pub. No.: US 2023/0159964 A1
(43) Pub. Date: May 25, 2023

(54) METHOD TO PRODUCE BIOCATALYTICAL COMPOSITIONS

(71) Applicant: INOFEA AG, Muttenz (CH)

(72) Inventors: Patrick SHAHGALDIAN, Hegenheim (FR); Maria Rita CORRERO, Hegenheim (FR); Lars GROSSNIKLAUS, Basel (CH); Anna Maria TIMM, Freiburg (DE); Yves Victor René DUDAL, Zürich (CH); Philippe F.-X CORVINI, Leymen (FR)

(21) Appl. No.: 17/997,724

(22) PCT Filed: May 6, 2021
(86) PCT No.: PCT/EP2021/061960

§ 371 Date: Nov. 1, 2022

(30) Foreign Application Priority Data
May 7, 2020 (EP) 20173422.5

(51) Int. Cl. C12P 7/62; C12N 11/14; C12N 9/20

(52) U.S. Cl. CPC C12P 7/62; C12N 11/14; C12N 9/20; C12Y 301/01003

(57) ABSTRACT

The present invention relates to a method of producing a composition, the composition comprising a solid carrier, a functional constituent and a protective layer to protect the functional constituent.

### THE FIELD OF THE INVENTION

The present invention relates to a method of producing a composition, the composition comprising a solid carrier, a functional constituent and a protective layer to protect the functional constituent, the method comprising the following steps:

(a) providing a suspension of a solid carrier;

(b) immobilizing a functional constituent on the solid carrier;

(c) forming a protective layer on the surface of the solid carrier to protect the functional constituent immobilized on the solid carrier;

(d) isolating the solid carrier comprising the functional constituent protected by the protective layer from the suspension;

(e) re-suspending the solid carrier comprising the functional constituent protected by the protective layer in an organic solvent; and

(f) isolating the solid carrier comprising the functional constituent protected by the protective layer from the organic solvent suspension. The present invention also relates to a composition comprising a solid carrier, a functional constituent and a protective layer to protect the functional constituent obtainable by said method.

### BACKGROUND OF THE INVENTION

Proteins such as enzymes are frequently needed, e.g. in industrial applications, diagnostics or for therapeutic use. In order to stabilize the proteins and/or to provide resistance to various types of stresses it has been suggested in the prior art to immobilize the proteins on the surface of a carrier and to protect them with a layer of protective material. Such an approach has been described e.g. in WO2015/014888 which discloses a biocatalytical composition comprising a solid carrier, a functional constituent like an enzyme and a protective layer for protecting the functional constituent by embedding the functional constituent at least partially and a process to produce such biocatalytical composition. Nevertheless there is a need for providing an economical and easy-to-use system for protecting functional constituents like proteins or protein-type compounds, particularly enzymes, against unfavorable influences thereby maintaining or even increasing their biological activity. Thus, there is a need for a protection system, which allows for use of the protected protein or enzyme at high enzymatic activity.

### SUMMARY OF THE INVENTION

The present invention provides a method of producing a composition, the composition comprising a solid carrier, a functional constituent and a protective layer to protect the functional constituent, the method comprising the following steps:

(a) providing a suspension of a solid carrier;

(b) immobilizing a functional constituent on the solid carrier;

(c) forming a protective layer on the surface of the solid carrier to protect the functional constituent immobilized on the solid carrier;

(d) isolating the solid carrier comprising the functional constituent protected by the protective layer from the suspension;

(e) re-suspending the solid carrier comprising the functional constituent protected by the protective layer in an organic solvent; and

(f) isolating the solid carrier comprising the functional constituent protected by the protective layer from the organic solvent suspension. The present invention also provides a composition comprising a solid carrier, a functional constituent and a protective layer to protect the functional constituent obtainable by said method.

It has been surprisingly found that the activity of the immobilized enzymes as described in WO2015/014888 can be significantly increased by isolating the immobilized enzymes obtained according to the process described in WO2015/014888, re-suspending them in an organic solvent, and isolating them from the organic solvent.

### DETAILED DESCRIPTION OF THE INVENTION

The present invention relates to a method of producing a composition, the composition comprising a solid carrier, a functional constituent and a protective layer to protect the functional constituent, the method comprising the following steps:

(a) providing a suspension of a solid carrier;

(b) immobilizing a functional constituent on the solid carrier;

(c) forming a protective layer on the surface of the solid carrier to protect the functional constituent immobilized on the solid carrier;

(d) isolating the solid carrier comprising the functional constituent protected by the protective layer from the suspension;

(e) re-suspending the solid carrier comprising the functional constituent protected by the protective layer in an organic solvent; and

(f) isolating the solid carrier comprising the functional constituent protected by the protective layer from the organic solvent suspension. The present invention also relates to a composition comprising a solid carrier, a functional constituent and a protective layer to protect the functional constituent obtainable by said method.

For the purposes of interpreting this specification, the following definitions will apply and whenever appropriate, terms used in the singular will also include the plural and vice versa. It is to be understood that the terminology used herein is for the purpose of describing particular embodiments only and is not intended to be limiting.

The term “solid carrier” as used herein refers to a particle. Usually the solid carrier is a monodisperse particle or a polydisperse particle, preferably a monodisperse particle. The solid carrier usually comprises organic particles, inorganic particles, organic-inorganic particles, self-assembled organic particles, silica particles, gold particles, magnetic particles and titanium particles. The particle size of the solid carrier is usually between 1000 μm and 1 nm, preferably between 100 μm and 10 nm, particularly between 50 μm and 50 nm, more particularly between 1 μm and 100 nm.

The term “functional constituent” as used herein refers to a constituent which imparts to the solid carrier to which this functional constituent is added its characteristic, functional property. A functional constituent in the sense of the present invention is usually a protein e.g.

an enzyme, an antibody, or RNA which has catalytic activity. Thus, in case the “functional constituent” is an enzyme, which is the preferred functional constituent, the carriers comprising the enzyme are enzymatically active. The functional constituent is immobilized to the solid carrier. Preferably the functional constituent is covalently bound to the solid carrier, in particular it is covalently bound to the surface of the solid carrier.

The term “linker” as used herein refers to any linking reagents containing reactive ends, which are capable of binding to specific functional groups (e.g. primary amines, sulfhydryls, etc.) of the solid carrier and the functional constituent, respectively. Various types of linkers are known in the art, including but not limited to straight or branched-chain carbon linkers and polyether linkers. For example, a linker may be immobilized on a solid carrier e.g. on the silica surface as a carrier material and then the functional constituent may be bound to an unoccupied binding-site of the linker. Alternatively, the linker may firstly bind to the functional constituent and then the linker bound to the functional constituent may bind with its unoccupied binding-site to the solid carrier.

The term “protective layer” as used herein refers to a layer for protecting the functional constituent of the composition. The protective layer of the present invention is usually built with building blocks at least part of which are monomers capable of interacting with both each other and the immobilized functional protein. The protective layers are usually homogeneous layers where all functional constituents, e.g. all enzyme present in the protective layer is active in the same way. The protective layer covers fully the solid carrier and covers partially or fully the functional constituent. Thus the functional constituent is partially or fully embedded by the protective layer.

The term “partially embedded functional constituent” as used herein shall mean that the functional constituent e.g. the protein is not fully covered by the protective layer, thus, the functional constituent is not fully embedded in the protective layer. In one embodiment less than 50% of the functional constituent of interest are covered by the protective layer, though typically more at least 70% will be covered, thus improving protection of the functional protein. In a particularly preferred embodiment, at least 70%, particularly at least 80%, more particularly at least 90%, most particularly at least 95% of the functional constituent are covered by the protective layer.

The term “fully embedded functional constituent” as used herein shall mean that the functional constituent according to the invention is 100% covered by the protective layer, i.e. that also the active site is covered.

The term “organic solvent”, as used herein shall mean a carbon-based substance that is used to dissolve another substance or substances i.e. is used to re-suspend the solid carrier comprising the functional constituent protected by the protective layer in step e) of the present method.

Since an organic solvent is carbon-based, it always has at least one carbon atom in its chemical structure. An organic solvent will also always have at least one hydrogen atom. Organic solvents usually comprise organic polar protic solvents, organic polar aprotic solvents and organic non-polar solvents. Organic polar protic solvents are e.g. methanol, ethanol, n-propanol, isopropanol, butanol and larger alcohols, acetic acid, formic acid. Organic polar aprotic solvents are e.g. acetone, acetonitrile, tetrahydrofurane, dimethylformamide, pyridine. Organic non-polar solvents are e.g. ethyl-actetate, diethyl-ether, methyl-ethyl-ketone, pentane, hexane, heptane, cyclohexane, toluene, benzene and nitrobenzene.

In a first aspect the present invention provides a method of producing a composition, the composition comprising a solid carrier, a functional constituent and a protective layer to protect the functional constituent, the method comprising the following steps:

(a) providing a suspension of a solid carrier;

(b) immobilizing a functional constituent on the solid carrier;

(c) forming a protective layer on the surface of the solid carrier to protect the functional constituent immobilized on the solid carrier;

(d) isolating the solid carrier comprising the functional constituent protected by the protective layer from the suspension;

(e) re-suspending the solid carrier comprising the functional constituent protected by the protective layer in an organic solvent; and

(f) isolating the solid carrier comprising the functional constituent protected by the protective layer from the organic solvent suspension.

In one embodiment, the method further comprises step (g) drying the solid carrier comprising the functional constituent protected by the protective layer to remove the organic solvent.

Thus in a preferred embodiment the present invention provides a method of producing a composition, the composition comprising a solid carrier, a functional constituent and a protective layer to protect the functional constituent, the method comprising the following steps:

(a) providing a suspension of a solid carrier;

(b) immobilizing a functional constituent on the solid carrier;

(c) forming a protective layer on the surface of the solid carrier to protect the functional constituent immobilized on the solid carrier;

(d) isolating the solid carrier comprising the functional constituent protected by the protective layer from the suspension;

(e) re-suspending the solid carrier comprising the functional constituent protected by the protective layer in an organic solvent;

(f) isolating the solid carrier comprising the functional constituent protected by the protective layer from the organic solvent suspension; and

g) drying the solid carrier comprising the functional constituent protected by the protective layer to remove the organic solvent.

In one embodiment the solid carrier is selected from the group of organic particles, inorganic particles, organic-inorganic particles, self-assembled organic particles, silica particles, gold particles, magnetic particles and titanium particles and is preferably an inorganic particle, more preferably a silica particle, even more preferably a silica nanoparticle (SNP). The particle size is usually measured by measuring the diameter of the particles. In case the solid carrier is a monodisperse particle, the size is usually between 1000 μm and 1 nm, preferably between 100 μm and 10 nm, particularly between 50 μm and 50 nm, more particularly between 1 μm and 100 nm. In case the solid carrier is a polydisperse particle, the size is usually between 1000 μm and 1 nm, preferably between 100 μm and 10 nm, particularly between 50 μm and 50 nm.

Usually monodisperse particles or polydisperse particles, preferably monodisperse particles are used as solid carrier in the present invention. In a preferred embodiment the monodisperse particles are spherical monodisperse particles. In a further preferred embodiment the polydisperse particles are non-spherical polydisperse particles.

Suspension of the solid carrier in step a) of the present method can be e.g. in an aqueous solution like water or aqueous buffer. In one embodiment the aqueous solution is suspended in step a) in a solvent different from the solvent used in step e). In one embodiment the solid carrier is suspended in step a) in a polar protic solvent, preferably water or buffer, and in step e) the solid carrier comprising the functional constituent protected by the protective layer is re-suspended in an organic solvent selected from the group consisting of organic polar aprotic solvents and organic non-polar solvents. In a preferred embodiment, the solid carrier is suspended in an aqueous solution in step a), more preferably in water or aqueous buffer, even more preferably in aqueous buffer. Buffers which can be used in the method of the present invention are phosphate, piperazine-N,N′-bis(2-ethanesulfonic acid), 2-Hydroxy-3-morpholinopropanesulfonic acid, N,N-bis[2-hydroxyethyl]-2-aminoethanesulfonic acid), (3-(N-morpholino)propanesulfonic acid), 2-[[1,3-dihydroxy-2-(hydroxymethyl)propan-2-yl]amino]ethanesulfonic acid, 4-(2-hydroxyethyl)-1-piperazineethanesulfonic acid), 3-(N,N-Bis[2-hydroxyethyl]amino)-2-hydroxypropanesulfonic acid, N,N-Bis(2-hydroxyethyl)-3-amino-2-hydroxypropanesulfonic acid, N-[Tris(hydroxymethyl)methyl]glycine, Diglycine, 4-(2-Hydroxyethyl)-1-piperazinepropanesulfonic acid, N,N-Bis(2-hydroxyethyl)glycine, N-[Tris(hydroxymethyl)methyl]-3-aminopropanesulfonic acid, N-(1,1-Dimethyl-2-hydroxyethyl)-3-amino-2-hydroxypropanesulfonic acid. Preferably a phosphate buffer is used.

The immobilization of a functional constituent on the solid carrier in step b) of the present method is usually carried out by adding a solution of the functional constituent to the suspension of the solid carrier. In a preferred embodiment the immobilization of a functional constituent on the solid carrier is carried out by providing a suspension of the solid carrier and adding a solution of the functional constituent, wherein the suspension with the added solution of the functional constituent is incubated to allow the functional constituent e.g the enzyme to bind on the surface of the solid carrier.

In a preferred embodiment the surface of the solid carrier is at least partly modified to improve immobilization of the functional constituent on the solid carrier. In particular, the surface of the solid carrier is at least partly modified before the functional constituent is immobilized. The surface of the solid carrier can be at least partly modified by introducing a molecule as anchoring point for the functional constituent to the surface of the solid carrier. In a preferred embodiment the surface of the solid carrier is partly modified by introducing a molecule as anchoring point for the functional constituent to the surface of the solid carrier. Thus in a more preferred embodiment the surface of the solid carrier is modified by introducing a molecule as anchoring point for the functional constituent to the surface of the the solid carrier. Said molecule used as anchoring point may be further modified by inducing a chemical reaction of the molecule as anchoring point with a linker, preferably a bi-functional cross-linker. Said molecule as anchoring point is in particular an amine moiety; more particularly amino-silane, even more particularly 3-aminopropyltriethoxysilane (APTES).

In a further preferred embodiment the functional constituent is immobilized on the solid carrier by a linker, preferably a bi-functional cross-linker binding to the functional constituent and to the surface of the solid carrier.

In a further more preferred embodiment the functional constituent is immobilized on the solid carrier by at least partly modifying the surface of the solid carrier by introducing a molecule as anchoring point as described supra for the functional constituent and by using a linker, preferably a bi-functional cross-linker binding to the anchoring point and the functional constituent of the solid carrier.

In one embodiment the introduced molecule as anchoring point and/or the linker are homogeneously distributed on the surface of the solid carrier.

In a preferred embodiment the bi-functional cross-linker is selected from the group consisting of glutaraldehyde, disuccinimidyl tartrate, bis[sulfosuccinimidyl]suberate, ethylene glycolbis(sulfosuccinimidylsuccinate), dimethyl adipimidate, dimethyl pimelimidate, sulfosuccinimidyl (4-iodoacetyl) aminobenzoate, 1,5-difluoro-2,4-dinitrobenzene, activated sulfhydrils, sulfhydryl-reactive 2-pyridyldithiol, BSOCOES (Bis[2-(succinimidooxycarbonyloxy)ethyl]sulfone), DSP (Dithiobis[succinimidyl]propionate]), DTSSP (3,3′-Dithiobis[sulfosuccinimidyl]propionate]), DTBP (Dimethyl 3,3′-dithiobispropionimidate2 HCl), DST (Disuccinimidyl tartarate), Sulfo-LC-SMPT (4-Sulfosuccinimidyl-6-methyl-a-(2-pyridyldithio)toluamido]hexanoate)), SPDP (N-Succinimidyl 3-(2-pyridyldithio)-propionate), LC-SPDP (Succinimidyl 6-(3-[2-pyridyldithio]-propionamido)hexanoate), SMPT (4-Succinimidyloxycarbonyl-methyl-a-[2-pyridyldithio]toluene), DPDPB (1,4-Di-[3′-(2′-pyridyldithio)-propionamido]butane), DTME (Dithio-bismaleimidoethane), BMDB (1,4 bismaleimidyl-2,3-dihydroxybutane). More preferably said bi-functional cross-linker is selected from glutaraldehyde, disuccinimidyl tartrate, disuccinimidyl suberate, bis[sulfosuccinimidyl] suberate, ethylene glycolbis(sulfosuccinimidylsuccinate), dimethyl adipimidate, dimethyl pimelimidate, sulfosuccinimidyl (4-iodoacetyl) aminobenzoate, 1,5-difluoro-2,4-dinitrobenzene, activated sulfhydrils (e.g. suflhydryl-reactive 2-pyridyldithio). Most preferred is glutaraldehyde.

In one embodiment the functional constituent is immobilized on the solid carrier in random orientation.

In one embodiment, the size ratio of solid carrier to functional constituents is such that it allows binding of between 10 to 10000, preferably of between 50 to 5000, more preferably of between 100 to 1000 functional proteins per particle.

The composition of the present invention is usually produced in a reaction vessel like a reactor. The formation of the protective layer according to step (c) of the present method is usually carried out by forming the respective protective layer with building blocks, wherein the building blocks build the protective layer in a polycondensation reaction. The polycondensation is usually effected in solution, preferably in aqueous solution. The polycondensation is usually effected in the suspension of the solid carrier comprising the functional constituent protected by the protective layer. Polycondensation can be easily controlled and stopped if appropriate, allowing that a defined thickness of the protective layer is achieved.

As building blocks for the protective layer usually structural building blocks and protective building blocks are used to build the protective layer. Preferably the protective layer is formed by building blocks, wherein as building blocks structural building blocks and protective building blocks are used to form the protective layer, wherein the structural building blocks are precursors of inorganic silica, capable of forming 4 covalent bonds in the layer formed and the protective building blocks are organosilanes. Structural building blocks which can be used are e.g. tetraethylorthosilicate (TEOS). Protective building blocks which can be used are e.g. 3-Aminopropyltriethoxysilane (APTES), n-Propyltriethyoxysilane (PTES), Isobutyltriethoxysilane (IBTES), Hydroxymethyltriethoxysilane (HTMEOS), Benzyltriethoxysilane (BTES),

Ureidopropyltriethoxysilane (UPTES), Carboxyethyltriethoxysilane (CETES). Structural building blocks are usually precursors of inorganic silica, capable of forming 4 covalent bonds in the layer formed. Protective building blocks are usually organosilanes, bearing an organic moiety endowed with the ability to interact with the functional constituents (e.g., enzyme). Preferred structural building blocks are tetravalent silanes, in particular tetra-alkoxy-silanes. Preferred protective building blocks are trivalent silanes, in particular tri-alkoxy-silanes e.g. 3-Aminopropyltriethoxysilane (APTES), n-Propyltriethyoxysilane (PTES), butyltriethoxysilane (IBTES), Hydroxymethyltriethoxysilane (HTMEOS),

Benzyltriethoxysilane (BTES), Ureidopropyltriethoxysilane (UPTES), Carboxyethyltriethoxysilane (CETES). Most preferred structural building blocks are mixtures of tetravalent silanes and trivalent silanes, in particular mixtures of tetra-alkoxy-silanes and tri-alkoxy-silanes. Particular preferred structural building blocks are selected from the group consisting of tetraethylorthosilicate, tetra-(2-hydroxyethyl)silane, and tetramethylorthosilicate. Particular preferred protective building blocks are selected from the group consisting of carboxyethylsilanetriol, benzylsilanes, propylsilanes, isobutylsilanes, n-octylsilanes, hydroxysilanes, bis(2-hydroxyethyl)-3 -aminopropylsilanes, aminopropylsilanes, ureidopropylsilanes, (N-Acetylglycyl)-3-aminopropylsilanes, in particular selected from benzyltriethoxysilane, propyltriethoxysilane, isobutyltriethoxysilane, n-octyltriethoxysilane, hydroxymethyltriethoxysilane, bis(2-hydroxyethyl)-3 -aminopropyltriethoxysilane, 3-Aminopropyltriethoxysilane, ureidopropyltriethoxysllane, (N-Acetylglycyl)-3-aminopropyltriethoxysilane, or selected from benzyltrimethoxysflane, propyltrimethoxysilane, isobutylimethoxysilane, n-octyltrimethoxysilane, hydroxyrnethyltrimethoxysilane, bis(2-hydroxyethyl)-3-aminopropyltrimethoxysilane, aminopropyltrimethoxysilane, ureidopropyltrimethoxysilane (N-Acetylglycyl)-3-aminopropyltrimethoxysilane or selected from benzyltrihydroxyethoxysilane, propyltrihydroxyethoxysilane, isobutyltrihydroxyethoxysilane, n-octyltrihydroxyethoxysilane, hydroxymefilyltrihydroxyethoxysilane, bis(2-hydroxyethyl)-3-aminopropyltrihydroxyethoxysilane, aminopropyltrihydroxyethoxysilane, Ureidopropyltrihydroxyethoxysilane (N-Acetylglycyl)-3-aminopropyltrihydroxymethoxysilane.

A particular preferred building block is TEOS as structural building block and APTES and/or hydroxymethyltriethoxysilane, preferably APTES as protective building block. In particular TEOS as structural building block and APTES as protective building block are used to build the protective layer. The reaction is usually carried out for a time period of between 0.5 to 10 hours, preferably between 1 and 5 hours, more preferably between 1 and 2 hours, preferably in aqueous solution and preferably at a temperature of about 5 to about 15° C. or at about 10° C. The formation of the protective layer can be stopped by actively stopping the polycondensation reaction e.g by removing the non-reacted building blocks e.g. by a washing step or by self-stopping of the polycondensation reaction caused by a limited amount of building blocks.

In some embodiments the protective layer has a defined thickness of about 1 to about 100 nm, preferably about 1 to about 50 mm, more preferably about 1 to about 30nm, even more preferably about 1 to about 25 nm, in particular about 1 to about 20 nm, more particular about 1 to about 15 nm. In a preferred embodiment, the protective layer has a thickness of about 1 to about 30nm, even more preferably about 1 to about 25 nm, in particular about 1 to about 20 nm, more particular about 1 to about 15 nm. The protective layer is usually porous and the pore size is between 1 and 100 nm, preferably between 1 and 20 nm.

The protective layer thickness can be measured, by using a microscope such as scanning electron microscope (SEM), transmission electron microscopy (TEM), scanning probe microscopy (SPM), light scattering methods or by ellipsometry.

In a preferred embodiment the functional constituent is a protein or RNA which has catalytic activity enzyme, more preferably an enzyme, an antibody, or RNA which has catalytic activity enzyme, even more preferably an enzyme, most preferably an enzyme selected from the group consisting of oxidoreductases, transferases, hydrolases, lyases, isomerases or ligases. Particular preferred is a hydrolase, more particular a lipase, even more particular a candida antarctica lipase B (CALB).

In one embodiment the protective layer embeds between 10% and 100%, preferably between 20% and 100%, more preferably between 30% and 100%, even more preferably between 50% and 100%, in particular between 70% and 100% of the functional constituent.

In a preferred embodiment the organic solvent is selected from the group consisting of organic non-polar solvents and organic polar aprotic solvents, in particular the organic solvent is selected from the group consisting of organic non-polar solvents and organic polar aprotic solvents, wherein the organic non-polar solvent is selected from the group consisting of ethyl-actetate, diethyl-ether, methyl-ethyl-ketone, pentane, hexane, heptane, cyclohexane, toluene, benzene and nitrobenzene and wherein the organic polar aprotic solvent is selected from the group consisting of acetone, acetonitrile, tetrahydrofurane, dimethylformamide, and pyridine, more particular an organic solvent selected from the group consisting of acetone, benzene, toluene, acetonitrile, pyridine and heptane, even more particular an organic solvent selected from the group consisting of acetone and heptane. More preferably the organic solvent is an organic polar aprotic solvent, even more preferably an organic polar aprotic solvent selected from the group consisting of acetone, acetonitrile, tetrahydrofurane, dimethylformamide, and pyridine, with acetone being most preferred. Equally more preferably the organic solvent is an organic non-polar solvent, even more preferably an organic non-polar solvent selected from the group consisting toluene, benzene and heptane, with heptane being most preferred.

In one embodiment, the polarity of the solid carrier comprising the functional constituent protected by the protective layer isolated from the suspension in step (d) may be modified prior to step (e). Modification can occur by incubating the particles with an non-polar organosilane. During this step the final diameter of the particle will not change. Thus in one embodiment the method comprises the following steps:

(a) providing a suspension of a solid carrier;

(b) immobilizing a functional constituent on the solid carrier;

(c) forming a protective layer on the surface of the solid carrier to protect the functional constituent immobilized on the solid carrier;

(d) isolating the solid carrier comprising the functional constituent protected by the protective layer from the suspension;

(e) re-suspending the solid carrier comprising the functional constituent protected by the protective layer in an organic solvent; and

(f) isolating the solid carrier comprising the functional constituent protected by the protective layer from the organic solvent suspension,

wherein the solid carrier comprising the functional constituent protected by the protective layer isolated from the suspension in step (d) is modified prior to step (e), preferably modified by incubating the solid carrier comprising the functional constituent protected by the protective layer isolated from the suspension in step (d) with an non-polar organosilane prior to re-suspending the solid carrier comprising the functional constituent protected by the protective layer in an organic solvent in step (e).

In a preferred embodiment the method comprises the following steps:

(a) providing a suspension of a solid carrier;

(b) immobilizing a functional constituent on the solid carrier;

(c) forming a protective layer on the surface of the solid carrier to protect the functional constituent immobilized on the solid carrier;

(d) isolating the solid carrier comprising the functional constituent protected by the protective layer from the suspension;

(e) re-suspending the solid carrier comprising the functional constituent protected by the protective layer in an organic solvent, wherein the organic solvent is an organic non-polar solvent, preferably an organic non-polar solvent selected from the group consisting of ethyl-actetate, diethyl-ether, methyl-ethyl-ketone, pentane, hexane, heptane, cyclohexane, toluene, benzene and nitrobenzene, more preferably selected from the group consisting of toluene, benzene and heptane, even more preferably heptane; and

(f) isolating the solid carrier comprising the functional constituent protected by the protective layer from the organic solvent suspension,

wherein the solid carrier comprising the functional constituent protected by the protective layer isolated from the suspension in step (d) is modified prior to step (e), preferably modified by incubating the solid carrier comprising the functional constituent protected by the protective layer isolated from the suspension in step (d) with an non-polar organosilane prior to re-suspending the solid carrier comprising the functional constituent protected by the protective layer in an organic solvent in step (e).

The non-polar organosilane used to incubate the particles prior to step (e) is usually selected from the group consisting of octyltriethoxysilane, benzyltriethoxysilane and butyltriethoxysilane, and is preferably octyltriethoxysilane. The solid carrier comprising the functional constituent protected by the protective layer isolated from the suspension in step (d) is usually incubated with an non-polar organosilane prior to step (e) between 0.1 to 5 hours, preferably between 0.5 to 2 hours.

The solid carrier comprising the functional constituent protected by the protective layer is usually isolated from the suspension in step (d) of the present method by centrifugation. The solid carrier is collected as a pellet and the supernatant is discarded.

The solid carrier comprising the functional constituent protected by the protective layer is usually re-suspended in an organic solvent in step (e) of the present method by pipetting up and down at least 10 times. Usually, the re-suspended particles are incubated in the organic solvent between 5 to 48 hours, preferably 10 to 20 hours, preferably at a constant temperature between 2 to 25° C., more preferably at a constant temperature between 15 to 25° C., even more preferably at a constant temperature of around 20° C.

The solid carrier comprising the functional constituent protected by the protective layer is usually isolated from the organic solvent suspension in step (f) of the present method by centrifugation.

Optionally the method further comprisies the step (g) drying the solid carrier comprising the functional constituent protected by the protective layer to remove the organic solvent. The solid carrier is usually dried by rotary evaporation under mild conditions or drying with a speed-vac system.

In a further aspect the present invention provides a composition comprising a solid carrier, a functional constituent and a protective layer to protect the functional constituent obtainable by the method as described supra. Solid carrier, functional constituent and protective layer are as described supra.

In one embodiment the present invention provides the use of the composition in a catalytic process, in particular the use of the composition in a catalytic process, wherein an esterification reaction is catalyzed by the composition. In particular, it is possible to use the composition of the invention in a catalytic process wherein during the process the composition is subject to at least one of a pH different from the optimal pH of the functional constituent in particular such that the pH value differs at least by +/−0.5 pH units and/or up to +/−5 pH units from the pH optimal for the functional constituent and/or to chemical stresses; and/or to biological stresses; and/or to solvents; and/or to physical stress; and/or to elevated temperatures, which exceed the optimal temperature for the functional constituent by at least 5° C.; and/or up to 60° C., particularly by 50° C., particularly by 40° C. higher, particularly by 30° C., particularly by 20° C., particularly by 10° C.; and/or to reduced temperatures, which deviate from the optimal temperature for the func-tional constituent by at least 5° C.; and/or up to 60° C.

### EXAMPLES

### Example 1: Candida Antarctica Lipase B (CALB) Immobilization on a Solid Carrier Material and Protection by an Organo-Silica Layer

CALB (EC 3.1.1.3) immobilization on a solid carrier material such as silica nanoparticles (SNPs) and protection can be carried out according to the following steps:
i. Surface modification of the SNPs in order to introduce anchoring points (i.e. amine) for the further chemical coupling with the enzyme
ii. Chemical reaction of the introduced amine moieties with a bi-functional cross-linker (e.g. glutaraldehyde)
iii. Enzyme coupling at the surface of the SNPs through the free active functions of the bi-functional cross-linker
iv. Polycondensation of silane building-blocks around both immobilized enzymes and free surface of the SNPs to yield a protective layer.

This synthetic procedure allows producing a protective layer at the surface of the SNPs surrounding and thus protecting the enzyme. The thickness of the produced protective layer can be adjusted by design, depending on the targeted application.
i) SNPs were produced using the conventional Stöber method adapted from the report of Imhof et al. (J. Phys. Chem. B 1999, 103, 1408), as follows. Ethanol (345.4 ml), ammonia 25% (39.3 ml) and TEOS (tetraethylorthosilicate, 15.3 ml) were mixed in a round bottom flask and this mixture was stirred at 600 rpm during 20 hours, at a constant temperature of 20° C. The resulting precipitate was consequently washed twice with ethanol and twice with water, and freeze-dried to yield bare SNPs that were characterized using scanning electron microscopy (Zeiss, SUPRA 40 VP). The acquired micrographs were used for particle size measurement using the analysis® (Olympus) software package (statistical analysis carried out on 100 measurements).
ii) In order to introduce, at the surface of the SNPs, amine functions that allowed the further anchoring of the to-be-protected enzyme, they were reacted with an amino-silane. It is important to note that this modification should be only partial so as to leave silanol groups for the further attachment of the protective layer.

In more details, SNPs in suspension in water (20 mL; 10 mg/ml) were incubated with APTES (3-aminopropyltriethoxysilane, 33 mg) during 90 minutes at 20° C. After two washing steps in water, the resulting amino-modified SNPs were reacted during 30 minutes with a bi-functional cross-linker (to allow the further immobilization of the enzyme), glutaraldehyde, at a final concentration of 1 g/L.
iii) After two washing steps in water, the resulting SNPs were re-suspended in a KPi (potassium phosphate) buffer (pH 6, 10 mM) at a final concentration of 10 mg/mL and incubated for 1 hour at 20° C. with the enzyme, CALB, (6 mg/ml) under magnetic stirring at 400 rpm.

The protection of the enzyme immobilized on SNPs was carried out by incubating the produced enzyme-immobilized SNPs with a mixture of silane building blocks that self-assembled around the enzyme and underwent a polycondensation reaction that created a protecting layer around the enzyme. The polycondensation reaction also occurred at the bare surface of the SNPs allowing the attachment of this layer at the surface of the SNPs. To that end, enzyme-immobilized SNPs (20 mL; 10 mg/ml) were first reacted at 20° C. under stirring at 400 rpm with 356 μl of TEOS. After 1 hour of reaction, 71 μl of APTES were added and the protective layer was allowed to grow at 10° C. for 150 minutes. Samples of SNPs were collected by centrifugation. Particles were washed twice in acetone, resuspended in acetone and incubated for 12 hours at 20° C. A control sample of shielded CalB was washed twice in buffer and incubated for 12 hours at 20° C. in KPi buffer.

After incubation, the particles which were incubated in acetone were collected and dried by means of a rotary evaporator, and stored at 4° C. The catalytic activity of the enzyme prior to collection and after incubation in acetone was measured using an activity assay with a chromogenic artificial substrate which is the 4-nitrophenyl butyrate (NPB). In brief, the NPB is hydrolyzed into p-nitrophenol, which can be measured spectrophotometrically at 415 nm. The enzymatic activity was measured as μmol of p-nitrophenol produced per minute. Enzyme activity of the immobilized enzyme after incubation in acetone was five time higher than the activity of the particles which were incubated in KPi buffer.

### Example 2: Improvement of the Esterification Reaction Catalyzed by an Immobilized and Shielded Candida Antarctica Lipase B (CALB)

CALB (EC 3.1.1.3) was immobilized on silica nanoparticles (SNPs) and protected following the general procedure described in Example 1. In more details, amino modified SNPs (10 mg/mL) were crosslinked with glutaraldehyde (final concentration 1 g/L). After extensive washes in water to remove unreacted material, the particles were resuspended in potassium phosphate buffer (10 mM, pH 6) and CalB (6 mg/ml) was added. The particles suspension was incubated in presence of the enzyme for 1 h at 20° C. under magnetic stirring at 400 rpm. Enzyme-immobilized SNPs (20 mL; 10 mg/ml) were reacted for 1 h with 356 μl of TEOS at 20° C. under stirring at 400 rpm. After, 71 μl of APTES were added and the layer was allowed to polycondensate for 150 minutes at 10° C. under magnetic stirring at 400 rpm.

The suspension of SNPs was washed three times in phosphate buffer (10 mM, pH 6). The particles suspension was split into two samples. One sample was incubated in potassium phosphate buffer (10 mM pH 7) for 12 hours at 20° C. and then stored at 4° C. The other sample was flash-frozen in liquid nitrogen and dried overnight in a lyophilizator. The obtained dried particles were resuspended in n-heptane and incubated with n-Octyltriethoxysilane (4.25 μL/mL of particle suspension), for 1 hour at 20° C. under 400 rpm stirring. After three washes in n-heptane, the particles were resuspended in n-heptane and incubated for 12 hours at 20° C. After incubation at 20° C. for 12 hours, particles were stored at 4° C.

The esterification reaction of butyric acid with n-propanol, catalyzed by CALB was measured by using a spectrophotometric assay which uses a pH indicator, the phenol red. The assay is based on the increase in absorption of phenol red (at 550 nm) as the pH value rises.

The increase of the pH corresponds to the decrease of free butyric acid and the formation of propyl butyrate. The reaction was measured in continuous at 30° C. and under orbital shaking (237 rpm) for 24 h. Esterification activity of the shielded CALB which was incubated for 12 hours in heptane was six times higher than the activity of the shielded CALB which was incubated for 12 hours in buffer.

1. A method of producing a composition, the composition comprising a solid carrier, a functional constituent and a protective layer to protect the functional constituent, the method comprising the following steps:
(a) providing a suspension of a solid carrier;
(b) immobilizing a functional constituent on the solid carrier;
(c) forming a protective layer on the surface of the solid carrier to protect the functional constituent immobilized on the solid carrier;
(d) isolating the solid carrier comprising the functional constituent protected by the protective layer from the suspension;
(e) re-suspending the solid carrier comprising the functional constituent protected by the protective layer in an organic solvent; and
(f) isolating the solid carrier comprising the functional constituent protected by the protective layer from the organic solvent suspension.

2. The method according to claim 1, further comprising the step
(g) drying the solid carrier comprising the functional constituent protected by the protective layer to remove the organic solvent.

3. The method according to claim 1 or 2, wherein in step (a) the solid carrier is suspended in water or aqueous buffer.

4. The method according to anyone of claims 1-3, wherein the protective layer is formed by building blocks, wherein as building blocks structural building blocks and protective building blocks are used to form the protective layer, wherein the structural building blocks are precursors of inorganic silica, capable of forming 4 covalent bonds in the layer formed and the protective building blocks are organosilanes.

5. The method of anyone of claims 1-4, wherein the functional constituent is a protein, preferably an enzyme.

6. The method of anyone of claims 1-5, wherein the protective layer embeds between 50% and 100% of the functional constituent.

7. The method according to anyone of claims 1-6, wherein the polarity of the solid carrier comprising the functional constituent protected by the protective layer isolated from the suspension in step (d) is modified prior to step (e).

8. The method according to anyone of claims 1-7, wherein the organic solvent is selected from the group consisting of organic non-polar solvents and organic polar aprotic solvents.

9. The method according to anyone of claims 1-7, wherein the organic solvent is an organic polar aprotic solvent.

10. The method according to claim 9, wherein the organic polar aprotic solvent is acetone.

11. The method according to anyone of claims 1-10, wherein the solid carrier is selected from the group of organic particles, inorganic particles, organic-inorganic particles, self-assembled organic particles, silica particles, gold particles, magnetic particles and titanium particles.

12. The method according to anyone of claims 1-11, wherein the suspension obtained in step (e) is incubated for 5 to 48 hours, preferably 10 to 25 hours, prior to isolating the solid carrier comprising the functional constituent protected by the protective layer from the organic solvent suspension according to step (f).

13. A composition comprising a solid carrier, a functional constituent and a protective layer to protect the functional constituent obtainable by the method according to anyone of claims 1-12.

14. The use of the composition of claim 13 in a catalytic process.

15. The use of the composition of claim 14, wherein an esterification reaction is catalyzed by the composition.
